(19) United States
(12) Patent Application Publication
(10) Pub. No.: US 2019/0362858 A1
(43) Pub. Date: Nov. 28, 2019

(54) SYSTEMS AND METHODS FOR MONITORING REMOTELY LOCATED INDIVIDUALS

(71) Applicant: GoFind Inc., Richardson, TX (US)

(72) Inventors: Anthony Valentino, Richardson, TX (US); Curt Terwilliger, Hillsborough, CA (US); Simon Milner, San Jose, CA (US); Ronald Kasper, Los Gatos, CA (US)

(21) Appl. No.: 16/421,875

(22) Filed: May 24, 2019

Related U.S. Application Data
(60) Provisional application No. 62/676,803, filed on May 25, 2018.

(51) Int. Cl. G16H 80/00; G16H 10/20; A61B 5/00

(52) U.S. Cl. CPC G16H 80/00; A61B 5/0022; G16H 10/20

(57) ABSTRACT

Systems and methods herein allow a monitored individual to monitored by a monitoring individual remote therefrom. The systems and methods allow the monitoring individual to define initial configuration settings by completing an on-line questionnaire. The monitored individual is then conditioned to use hardware components of the monitoring system based on these initial configuration settings, and then the system continues to self-learn and modify generated prompts to the monitored individual based on continued use and/or non-use of the hardware by the monitored individual.

### CROSS-REFERENCE TO RELATED APPLICATIONS

This application benefits from and claims priority to U.S. Provisional Application No. 62/676,803, filed May 25, 2018, which is incorporated by reference in its entirety herein.

### BACKGROUND

The caring for aging yet still independent parents has increased in complexity in the 21^{st} century as geographic dispersion of the family unit has increased. As late as the mid 20^{th} century, family units were still concentrated geographically such that daily or weekly visits by the children to their parents could not only provide the parent with assistance in tasks that either could no longer be performed by the parent or could only be performed with great difficulty, but also provide the parent with much needed companionship as the parents' mobility decreased with advancing age.

As the century drew to a close, the family unit dispersed for a number of reasons such as pursuing better economic opportunity or pursuing a change in lifestyle. Advanced telecommunications technology such as email, Social Media platforms, SMS, etc. was used to try to bridge the gap with varying degrees of success. For example, people who have immigrated to the US from India daily attempt to check in with their parents via SMS messages if only to just confirm that all is well in both places. The millions of SMS messages can sometimes overwhelm the telecom infrastructure in both locations leading to some frustration and concern for the parent's wellbeing. In addition, utilizing SMS or Social Media for these interactions is still somewhat impersonal . . . .

### SUMMARY OF THE EMBODIMENTS

The embodiments described herein detail a novel system architecture for automatic, daily parent engagement via Internet-connected devices and software, to provide a more personal interaction to the aging parent. ‘Parent’ is one use case. The system described herein may apply to any individual under care.

Embodiments herein provide the advantage that aging may stay in a family home as opposed to assisted living by identifying to others (e.g., loved ones, children, etc.) that the aging person is safe and keeping mentally and physically active, even if the aging person is living independently. Embodiments described herein may provide benefits for autistic children, and other categories of persons.

### BRIEF DESCRIPTION OF THE FIGURES

FIG. 1 depicts a system for connecting, communicating, and caring for remotely located individuals.

FIG. 2 depicts the monitor device in further detail, in embodiments.

FIG. 3 depicts example configuration settings, in embodiments.

FIG. 4 depicts an example use case of the system of FIG. 1 and a method implemented thereby as described above with respect to FIGS. 1-3, in an embodiment.

FIG. 5 is a chart showing monitored individual interaction with the system according to the multi-parameter response function, in an example.

FIG. 6 depicts a method for monitoring an individual at a remote location, in an embodiment.

### DETAILED DESCRIPTION OF THE EMBODIMENTS

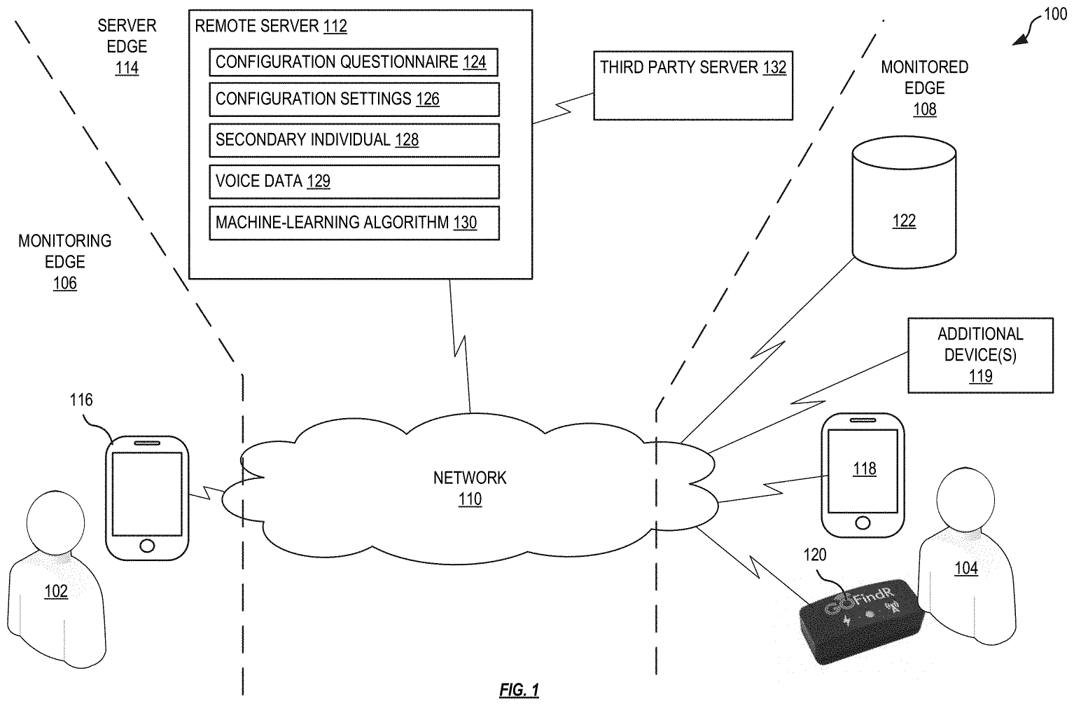

FIG. 1 depicts a system 100 for connecting, communicating, and caring for remotely located individuals. At a macro-level, system 100 allows a monitoring individual 102 to connect, communicate, monitor, and otherwise care for a monitored individual 104. Monitoring individual 102 may represent a single individual, or a plurality of individuals (independently or in concert) monitoring the monitored individual 104 without departing from the scope hereof. The monitoring individual 102 may be located at a monitoring edge 106 and the monitored individual 104 may be located at a monitored edge 108 and the connection, communication, monitoring, and otherwise caring for occurs via interaction with network 110. The monitoring edge 106 may represent a first geographical area and the monitored edge 108 may represent a second geographical area which may be the same, or different, from the first geographical area. For example, the first and second geographical area may be the state, city, workplace, and/or dwelling of the monitoring individual 102 and the monitored individual 104, respectively.

The network 110 may be any wired or wireless network, including but not limited to, USB, Ethernet, Wi-Fi, cellular, radio-frequency, or any other communication means. The network 110 may further be in communication with a remote server 112 that implements one or more back-end API and machine learning functions as discussed herein. The remote server 112 is schematically shown in FIG. 1 as located in a server edge 114, which may be at a third geographical area the same as or different from the first and/or second geographical area. The third geographical area may be the “cloud” in the sense of a remote computing environment accessible by one or more remote devices via the network 110. The remote server 112 includes at least one processor and memory storing computer readable instructions that, when executed by the processor operate to control the processor to implement the functionality of the server 112 discussed herein.

The monitoring individual 102 interacts with a first device 116 to connect with, communicate with, care for, and otherwise monitor the remotely located monitored individual 104. The first device 116 may be any device having a display, I/O interface (e.g. touch-screen display, keyboard and mouse, microphone, camera, etc.). Accordingly, the first device 116 may be one or more of a smartphone, tablet, laptop computer, desktop computer, smart TV, smart speaker, or the like. The first device 116 is in communication (either wired or wireless) with the network 110, as well as the other devices of system 100 in communication with the network 110. Further, the monitoring individual 102 may monitor a list or group of monitored individuals 104. The system may be configured so that monitoring individual can view the location of a single monitored individual in the list of monitored individuals while receiving an alert or notification from any one of the monitored individuals in the list.

The monitored individual 104 may interact with a second device 118 that is similar to and includes one or more of the above discussed above with respect to the first device 116. The monitored individual 104 may further interact with a monitor device 120 and a smart speaker 122.

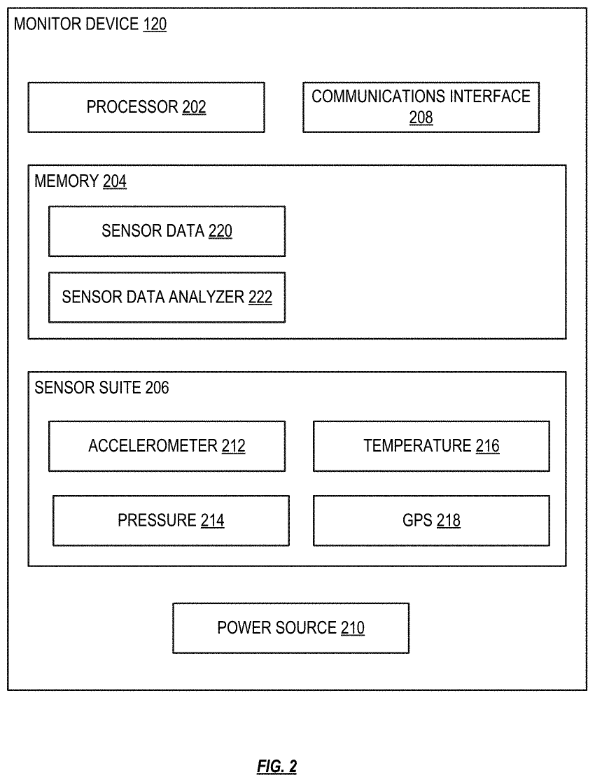

FIG. 2 depicts the monitor device 120 in further detail, in embodiments. The monitor device 120 may include a processor 202, a memory 204, a sensor suite 206, a communications interface 208, and a power source 210. The processor 202 may be any computing device or microprocessor capable of executing computer readable instructions stored in the memory 204 that implement the functionality of the monitored device discussed herein. The memory 204 may be volatile and/or non-volatile.

The sensor suite 206 may include one or more sensors that read information about the monitored individual 104 and/or the related area that the monitored individual 104 is located in. For example, the sensor suite 206 may include an accelerometer 212, a pressure sensor 214, a temperature sensor 216, and a GPS receiver 218. The sensor suite 206 may include other sensors as well, such as a hydration sensor, heartbeat sensor, other biometric sensor, and the like. Data captured by each respective one of the sensors in the sensor suite 206 may be stored in the memory 204 as sensor data 220.

The memory 204 may store a sensor data analyzer 222 as computer readable instructions that when executed by the processor 202 operate to analyze the sensor data 220 to determine signatures, within the sensor data 220, indicative of actions taken by the monitored individual 104. These signatures could include one or more of a gait, a step, a walk, a run, a location, geofence analysis, temperature, facial impression, heartbeat, and other sensor data signatures. It should be appreciated that, in other embodiments, the sensor data analyzer 222 is located remote to the monitor device 120, such as in the remote server 112, and the sensor data 220 is transmitted to the remote server 112 via communications interface 208 and network 110.

The monitor device 120 may include any one or more features discussed in U.S. Provisional Application 62/655,630 (including the appendices thereto), entitled Automatic Autonomous GeoFence Creation, and filed Apr. 10, 2018, which is incorporated by reference in its entirety.

The smart speaker 122 may be a device capable of prompting for, and receiving commands from the monitored individual 104, and receiving responses therefrom. For example, the smart speaker 122 may be an Amazon Echo®, Google® Home, Sonos® One, or the like. The smart speaker 122, in some embodiments, may additionally or alternatively be a display such that visual as well as audio interaction may occur with the monitored individual 104. The smart speaker 122, in some embodiments, may additionally or alternatively contain a voice recognition subsystem which allows the monitored individual's 104 verbal inputs to be acted upon by the system.

In certain embodiments, the system 100 learns the behavior of the monitored individual 104 and, within the operating framework defined by the system 100 or the monitoring individual 102, evolves in its understanding of the monitored individual 104 in their monitored-edge (e.g., monitored edge 108) and out-of-edge activity habits. Embodiments stimulate the monitored individual 104 both mentally and physically, resulting in the monitored individual 104 bonding and forming a relationship with the system 100 as a proxy for the relationship the monitoring individual 102 because of the distance between the monitoring individual 102 and monitored individual 104. The monitoring individual 102 may then receive updates or other prompts indicating that the monitored individual 104 is acting according to normal and expected behavior.

In some embodiments, the system 100 may need to be initially trained according to the monitored individual's 104 habits, and the monitoring individual's 102 desired configuration of the system 100. Accordingly, the remote server 112 may store a configuration questionnaire 124. The configuration questionnaire 124 may be accessed by one or more of the monitoring individual 102 and the monitored individual 104 via an application running on the first device 116 and the second device 118, respectively. The configuration questionnaire 124 may include a series of questions that will help generate configuration settings 126. For example, the configuration questionnaire 124 may query the monitored individual 102 as follows:
What is John Doe's age?
When should John Doe wake up at morning?
How much activity should John Doe participate in daily, and by what time?
How many steps per day should John Doe take?
Do you want to link John Doe's calendar?
What medications does John Doe take, and what dosages/amounts per day?
How much hydration intake do you want John Doe to take?
Do you want to schedule any automated home system actions?
When is John Doe's bedtime?
Does John Doe have any pre-existing health conditions?

Responses to these questions may then create a series of configuration settings 126. It should be appreciated that other questions and interactions may occur during the configuration questionnaire without departing from the scope hereof. For example, during the configuration questionnaire 124, it may be determined that third party, such as a neighbor or closest relative to the monitored individual 104 may also be notified of circumstances of the monitored individual 104. As such, the remote server 112 may further store a secondary individual list 128 that identifies third parties to be notified of certain instances of the monitored individual 104, as discussed below.

In certain embodiments, the monitoring individual 102 interact with the first device 116 to capture one or more voice recording 129 which are used to generate prompts to the monitored individual 104 as discussed below. One example of voice data 129 may be the situation where, each morning, any one or more monitoring individuals 102 sends a voice memo to the monitored individual 104, wishing all in the family a wonderful day. The anticipation of the monitored individual 104 builds over time and establishes a morning routine eventually incorporated and initiated by the monitored individual 104. A further example is an evening compilation of each of the monitored individual 104's through-the-day activities compiled into a voice memo for the monitored individual 104 in the voice of the monitoring individual 102.

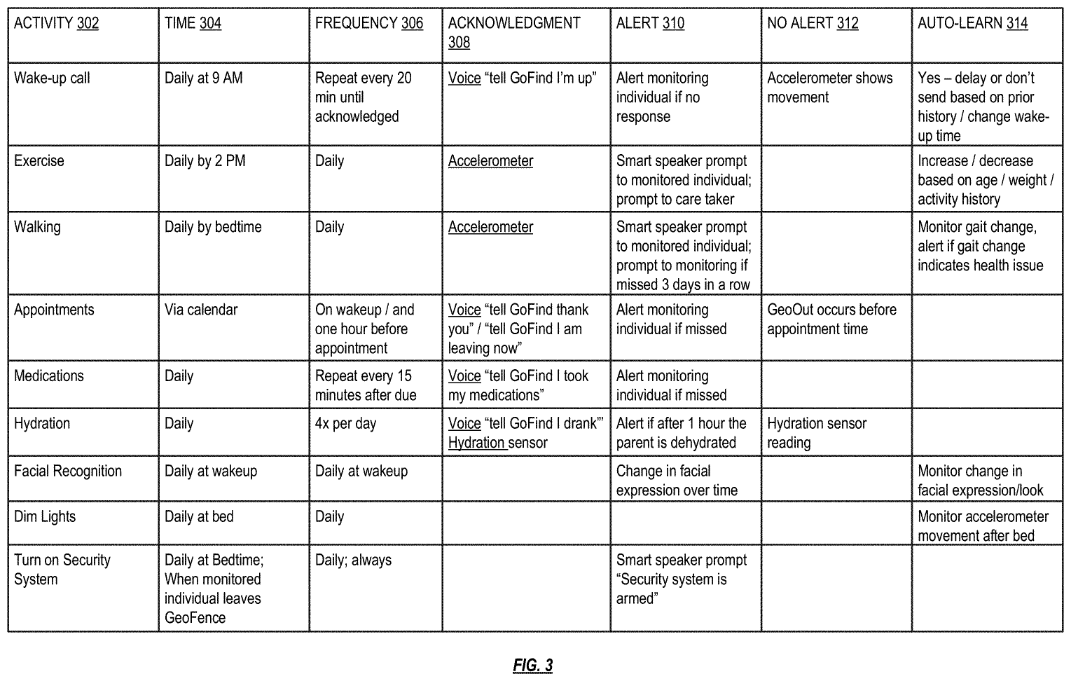

FIG. 3 depicts example configuration settings 126, in embodiments. The configuration settings 126 may include one or more of an activity 302, a time 304 for the activity 302 to occur, a frequency 306 for the activity 302 to occur, an acknowledgment 308 required for the system 100 to understand that the activity 302 has occurred, an alert 310 for the system 100 to generate when the activity 302 does not occur, a no-alert condition 312 that serves as a backup acknowledgement that the activity 302 has occurred, and an auto-learn determination 314 indicating how the system 100 will auto-learn based on the activity.

The activity 302 may define an action to be taken by the monitored individual 104, such as waking up, exercise, steps taken, medications, appointments, water intake, and facial recognition via a picture taken (which may be taken by the second device 118 or an alternative camera that is positioned at a common location—e.g., the bathroom mirror. The activity 302 may alternatively or additionally define an action to be taken with respect to the monitored edge 108, such as dimming lights or turning off/on of a security system. As such, it should be appreciated that the system 100 may include additional devices 119, such as connected home devices that allow for activities 302 to define interactions with the connected home devices. Examples include, but are not limited to:
Pre-programmed time-of-day mood settings
   Music, lighting, temperature—possibly, even scent
   Auto increase lights brightness, cooler temperature upon waking
   Auto dim lights, warmer temperature as bedtime approaches
   Wellness Devices
   Blood oxygenation measurement
   Blood pressure measurement
   Hydration patch measurement
   Pressure sensitive weekly medication pill box
   Weighing scale
   Camera(s) for capturing image of monitored individual 104
Home Entertainment
   TVs/video screens
   Audio speakers
   Game consoles tailored for the elderly, such as iPad with large font and graphics for computerized board games
Home Security that automatically secures the home at pre-set times and conditions
Monitored individual SoS devices. As an example, if the monitored individual 404 has fallen or is immobilized besides sending an alert from the accelerometer data the system may also command the connected home lights and sound siren systems to activate thus sending an alert to the neighbors.
Ordering online transportation: for example:
   The monitored individual 104 orders for themselves, or can be granted as part of reward system, or system automatically orders based on calendar event
   Pick up, drop off outbound, and again for return journey where the monitor device 120 acts as the pickup location pin for the third-party transportation
   Pre-programmed constraints as defined in the configuration questionnaire 124 such as monetary limits and geofence zones

It should be appreciated that these actions are only examples and other actions may be included without departing from the scope hereof

The time 304 and frequency 306 define when the action 302 is to occur, and how often to repeat the action 302, respectively. For example, the wakeup call action is to occur daily at 9 AM, and repeat every 20 minutes until the action 302 is acknowledged by the monitored individual 104. The time 304 does not need to be a clock-based time, but may also be a conditional or reactive definition. For example, the security system action may occur whenever the monitored individual 104 leaves a geofence defined by the system 100.

The acknowledgment 308 defines a response that the monitored individual 104 must give in order for the action 302 to be met. For example, the response may be a verbal acknowledgement that is spoken to the smart speaker 122. Additionally, or alternatively, the acknowledgment may be a data signature identified by the sensor data analyzer 222 by monitoring the sensor data 220.

The alert 310 defines a prompt to the monitored individual 104, the monitoring individual 102, or a third party, such as the police or the secondary monitoring individual 128. For example, the alert 310 may identify a prompt played over the smart speaker 122 to the monitored individual 104 indicating to take their medication. As another example, the prompt could be an SMS to one or more of the monitoring individual 102 when the walking goal is missed 3 days in a row. As another example, if the monitored individual 104 is not waking up, an SMS or phone call prompt could be sent to the monitoring individual 104 and the secondary individual 128 to check on the monitored individual 104.

The no alert 312 defines a condition where the alert 310 does not need to be sent, even if the acknowledgment 308 is not received. For example, if the accelerometer 212 captures movement, it is known that the monitored individual 104 is awake and moving and thus does not need to be checked on. As another example, if the GPS 218 identifies the monitored individual 104 as leaving and travelling towards the location of an appointment, the alert 310 does not need to be generated.

The auto-learn 314 identifies potential algorithms to be implemented by a machine learning algorithm 130 (FIG. 1) that is computer readable instructions that when executed by a processor operate to monitor the actions 302 over a period of time and determine characteristics of the monitored individual 104. For example, the machine learning algorithm 130 may monitor the acknowledgement 308 time over a period of days (or weeks, months, or any period of time) to determine a more appropriate wakeup time. In such an example, if the monitored individual 104 frequently wakes up before the 9 AM wake-up call, and data from the accelerometer 212 indicates that the monitored individual 104 frequently awakes and gets out of bed around 8 AM, the machine learning algorithm 130 may modify the time 304 of the wake-up call activity 302 to 8 AM instead of 9 AM.

As another example, the machine learning algorithm 130 may monitor the gait of the monitored individual 130 over time to determine potential health risks. For example, a healthy person may have a walking gait of around 22 to 30 inches, depending on the height, weight, etc. Once the initial gait threshold is set, the system may monitor the gate to determine a change over time, or a shuffling gait during walking. As another example, the machine learning algorithm 130 may monitor change in facial expression and/or look to identify potential health risks. As another example, the machine learning algorithm 130 may monitor the accelerometer data captured by accelerometer 212 to, while prompted to standup, stand still, and follow a deep breathing exercise, monitor for tremors to identify early onset for Parkinson's disease. As another example, the machine learning algorithm 130 can monitor interaction with the smart speaker 122 to identify slurring in the monitored individual 104's voice.

The machine learning algorithm 130 may monitor the monitored individual's 104 interaction with the monitor device 120. In such instances, if the monitored individual 104 removes the monitor device 120 on a consistent basis (such as before bed or before taking a shower), the machine learning algorithm 130 will interpret the lack of movement during these periods to be acceptable to the monitored individual 102. The system 100 may issue a verbal alert (e.g., via smart speaker 122) after a period of time to the monitored individual 104 to put the monitor device 120 back on. It will continue to send this alert, until either movement is detected by the accelerometer 212 of the monitor device 120, or the monitored individual 104 gives a verbal confirmation of putting the monitor device 120 on their body.

The machine learning algorithm 130 may also monitor interaction by the monitored individual 104 with the smart speaker 122 that is not specifically associated with one of the actions 302. In turn, this allows the system 100 to provide a lure-reward phase where the monitored individual 104 receives a prompt or other reward for consuming/acting upon a system stimulus. The machine learning algorithm 130 self learns the monitored individual 104's behavior and self qualifies stimuli that increases monitored individual 104 interaction with the system 100 through use of the second device 118, the monitor device 120, or the smart speaker 122. An example of a prompt or other reward may be identification of movies, books, songs, or other types of media that the monitored individual 104 listens to via the smart speaker 122. Upon identifying specific likes and dislikes of the monitored individual 104, the machine learning algorithm 130 may enable the system 100 to become a “companion” to the monitored individual 104 via unsolicited prompts/actions/questions are made to the monitored individual 104 via the smart speaker 122. Example stimulus that may be auto-learned include, but are not limited to:
Automatic Wake-up Calls
Delivery Service—“Package en-route”—builds the monitored individual 104's anticipation
Books
   Accessing the local library for books that are read to the monitored individual 104
Movies
   Access an on-line movie and play directly on a designated TV
News
   Accessing the monitored individual 104's favorite news channel(s). The machine learning algorithm 103 knows if the medium is audio or video, and automatically plays on the appropriate device in the monitored individual 104's home
Weather Forecast
   Today's local weather forecast
Podcast
   Accessing the monitored individual 104's favorite comedian
Music
   Stream pre-selected or related music. For example, the monitored individual 104's play list of Frank Sinatra songs.
Real-time or pre-saved monitoring individual 102 Voice Memo
   Real-time voice memos sent from monitoring individual 102 at any time, or memos saved as voice data 129—impromptu through the day personal messages
   May include images or videos—automatically displayed on connected home TV/video screens
Local Community Events and Invitations
   Events happening in the monitored individual 104's locality
   If the monitored individual 104 accepts the invitation, the System automatically schedules, reminds the monitored individual 104 as the ‘trip out’ is approaching, and orders online transportation service—with reminders and notifications to the monitored individual 104 at each stage in the process.
Medicine Alerts
Factoids Daily Update
Automatic Scorecard Daily Update
   Score out of 100 based on multi-parameter inputs and sliding time window
   Suggestions on how to improve daily Score
Automatic prompts—tied to a reward—include, but are not limited to the following:
   Standup for n number of preprogrammed minute(s)
   Walk for n number of preprogrammed minute(s)
   Standup, stand still and breathe deeply for n number of preprogrammed minute(s)
   Daily routines, with automatic prompts—tied to a reward—include, but are not limited to the following, where any number of the instrumentation devices below are part of the connected home network:
      Fixed-position every-day selfie picture
      Blood oxygenation measurement
      Blood pressure measurement
      Hydration patch measurement
      Pressure sensitive weekly medication pill box
      Weighing scale

The above discussed system and method allows for a “monitoring” and “monitored” use model. The monitoring primary use model is a mobile- and web-based push-pull configuration-notification. Configuration requires active monitoring individual 102 input into the system via completion of the configuration questionnaire 124. Monitoring individual 102 are notified in real-time, together with a web-based monitored individual 104 activity on the System dashboard.

The monitored primary use model is:
Seamless wearable and third-party hardware ecosystem sensing/monitoring devices requiring physical monitored individual 104 action
Voice-interface with monitored individual 104 participation via the smart speaker 122
Audio prompts to monitored individual 104 via the smart speaker 122
Audio content streamed to monitored individual 104 via the smart speaker 122 or another device (such as smart TV).

Certain embodiments include a backup, secondary use model providing emergency, direct voice communication between monitoring individual 102 and monitored individual 104, including secondary individual 128 automatic detection (e.g., via monitoring location of the secondary individual 128 and determining the closest secondary individual 128 to the monitored individual 104) and voice call activation for fastest response.

In certain embodiments, the remote server 112 is connected, either wired or wirelessly, to a third-party server 132. The third-party server 132 may provide third party cloud services managed by Google, Amazon or Apple, for example, such as Artificial Engines and Predictive Health Analytics data pipelines; Automated pick-up, transport, and delivery service; audio and visual media content; etc.

### EXAMPLES

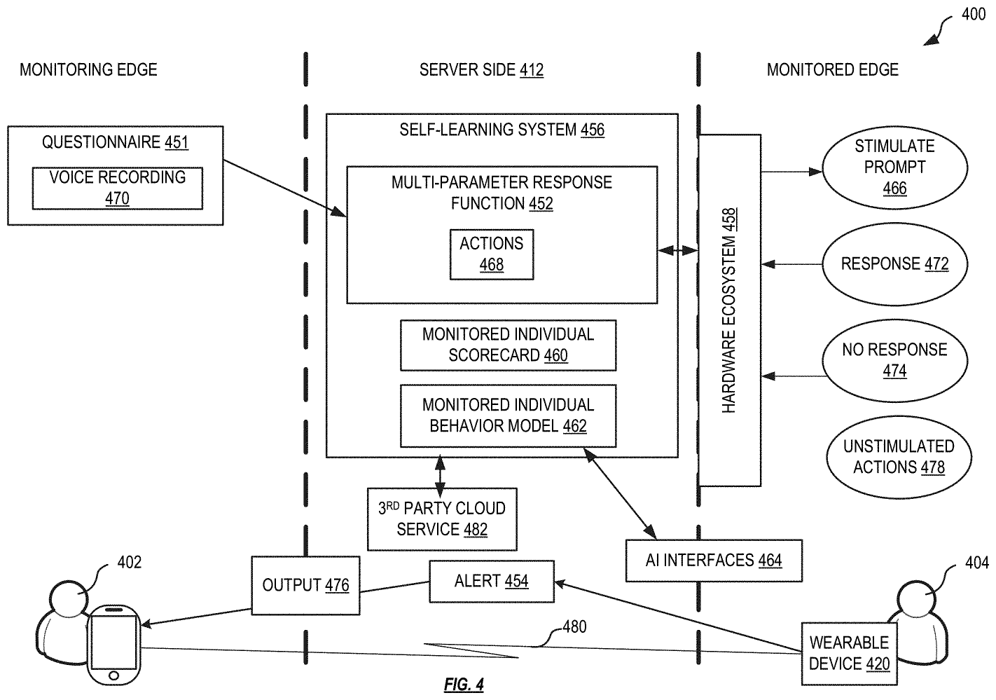

FIG. 4 depicts an example use case 400 of the system 100 and method implemented thereby as described above with respect to FIGS. 1-3, in an embodiment. The use case 400 describes a monitored individual 404 (e.g., the monitored individual 104), which may be a parent, and a monitoring individual 404 (e.g., the monitoring individual 102), which may be a caretaker, use case where the caregiver(s) may be a child or relative that is distant from the parent. The monitored individual 404 is primarily in a home environment. Use case 400 utilizes a wearable technology 420 (e.g., the monitor device 120) to monitor the monitored individual 404 and a cloud-based software located in the server 412 (e.g., the remote server 112) additionally provides tracking information on monitored individual 404 when outside the home. Thus, the server 412 includes at least one processor and memory storing computer readable instructions that, when executed by the processor operate to implement the functionality of server 412 discussed herein.

Use case 400 implements a lure-reward conditioning system that encourages the independent monitored individual 404 to interact with the system, establishing monitored individual 404 daily habits with system. Thereby, the monitored individual 404 and system bond, and form a relationship.

The system learns the behavior of the independent monitored individual 404 and, within the operating framework defined by the monitoring individual(s) 402, evolves in its understanding of the monitored individual 404 in their environment, stimulates both mentally and physically, resulting in the monitored individual 404 bonding and forming a relationship with the System.

Use case 400 begins at block 451 with each person designated as a monitored individual 402 filling out an online web- or App-based family questionnaire (which is an example of and like configuration questionnaire 124). Consensus must be achieved before this task is complete. Once the questionnaire is complete, the monitoring system is activated and the above discussed configuration settings 126 are created in the server 412 as a multi-parameter response function 452 and transmitted to the monitoring device 420. The multi-parameter response function 452 is an example of the configuration settings 126 of FIG. 1, discussed above, and defines intended interactions of the monitored individual 404 with the system (such as hardware ecosystem 458, discussed below).

In an example, the questionnaire 451 is a matrix, where each cell in the matrix is parameterized. Examples of the questionnaire allow the monitoring individual 102 to decide which activated sensors/monitoring devices are interactable with the monitored individual 404, and/or which events are monitored by the system. These activated sensors, or combination thereof, must be activated to create a System-internal event (e.g., activity 302 discussed above). Each event has a time-to-respond (e.g., time 304 discussed above) and a timeout configurable parameter. This may be implemented as a Wizard, with default pre-set values.

The server 412, and the wearable device 420 then operates according to the multi-parameter response function 452 (such as under control of configuration settings 126) and uses sensors to measure the monitored individual's 404's activity. For example, if the monitored individual 404 is inactive for 2 hours or more, the system prompts the monitored individual 404 to get up and move. The sensors on the wearable device 420 (e.g., sensors in sensor suite 206) detect the movement and the system and the event is considered closed. If the sensors detect no movement, the prompt repeats. If the monitored individual 404 does not respond to the prompt, or repetition thereof, an alert 454 (e.g., alert 310) is sent to a monitoring individual 402 (e.g., via SMS or other communication protocol to the monitoring individuals electronic device) indicating no movement for several hours.

As another example of an event activatable in response to the questionnaire, the system may detect the monitored individual 404 has fallen (e.g., via detection of a fallen signature in the sensor data 220, discussed above). If the sensors of the wearable device 420 detect that the monitored individual 404 does not get up, the wearable device 420 transmits an SOS alert 454 that is sent directly to, or relayed via the server 412, to the monitoring individuals 102. Upon detection of a fallen signature in the sensor data 220, the system may wait for a verbal or gesture confirmation back from the fallen parent 404 (received at the wearable device 420, or at another device such as the smart speaker 122 of FIG. 1) indicating a confirmed-positive fallen state. In a fall event example, the following sequence of events facilitate a confirmation mechanism for a detected dangerous condition:
The system detects a possible monitored individual 404 fall based on sensor data 220 captured by the wearable device 420. This detection may occur within the wearable device 420 in which an indication thereof is transmitted to the server 412, or within the server 412 monitoring sensor data captured by the wearable device 420 and transmitted to the server 412.;
A verbal request for confirmation is initiated (e.g., via speaker 122 or via the wearable device 420);
The fallen monitored individual 404 can either confirm back to the system via a body gesture, using for example the GoFind, Inc. wearable device 420, or via a verbal confirmation back to a smart speaker (e.g., speaker 122), depending on the location of the fallen monitored individual 404.
   In this way, the fall event allows the system intelligence to seamlessly connect and communicate with the fallen monitored individual 404 through any combination of verbal input/out and physical gesture input/output into/out of the system.

As another example, a geo-fence use case in which the system detects that the monitored individual 404 has left a defined safe zone (e.g., via a geofence breach) and if the monitored individual 404 doesn't return to the safe zone within a predetermined amount of time (e.g., 30 seconds), an alert 454 is sent to the monitoring individual 402. The boundary of the geofence may be located in the configuration settings 126 of the multi-parameter response function 452.

The multi-parameter response function 452 shown in FIG. 4 may evolve in stages. It may begin in an initial state of configuration settings (e.g., configuration settings 126 of FIG. 1) in response to the monitoring individuals completing the questionnaire 451. A self-learning system 456 may then implement a conditioning stage in which the monitored individual 404 is conditioned to interact with the system. Lastly, the self-learning system 456 may enter a continued-learning stage in which the self-learning system 456 learns what combination of stimuli, together with time-of-day and geo proximity solicits maximum monitored user 404 interaction with wearable device 420, or external devices (e.g., smart speaker 122, second device 118, or additional devices 119). Thus, the self-learning system 456 is constantly self-tuning the multi-parameter response function 452 (and thus the configuration set to increase interaction.

For example, the system builds geo fences and learns that it is normal for the Parent to leave the house at noon to visit the coffee shop/grocery store—and, consequently, will not create an alert to the Caregiver(s).

The conditioning stage, as well as the continued-learning stages are examples of the machine learning algorithm 130 discussed above and may evolve the multi-parameter response function 451, interpreting across three axes, each axis containing multiple input/outputs:
the initial questionnaire 451, discussed above
The systems generation of a stimulus or non-stimulus event via the wearable device 420 and/or other devices in hardware ecosystem 458
and the monitored individual 404's response to the stimulus or non-stimulus event.

During implementation of the conditioning and continued-learning stages, the self-learning system 456 shown in FIG. 4 may maintain a monitored individual scorecard 460 that is a measure of monitored individual 404's sustained, daily engagement, including, but not limited to the following:
Score: Out of 100
Complete activity and possible Reward

Rewards include, but not limited to the following:
Online delivery of ‘treats’:
   “Congratulations, you've won a treat”
   “Congratulations, you've graduated to the next level”
   Online ordered and Fulfilled—from Self-Learning System—notification to Caregiver(s)
   Notifies Parent 1 hour prior to being delivered
Un-layering of Content
   Daily news
   Audio books: Short Stories; One Chapter/few pages streamed per day
   Messages from Caregiver(s)

The present application acknowledges that elderly users do not typically interact with electronics. As such, the scorecard 460 may be accessible by the monitored individual 404, via a web- or mobile-application (e.g., smartphone application) such that the monitored individual 404 is incentivized to interact with the wearable device 420 and/or other devices in the hardware ecosystem 458. Further the scorecard 460 may indicate progress towards a specific reward (which may be generic or set by the monitoring individual 402) thus further incentivizing the monitored individual 404 to interact with the system.

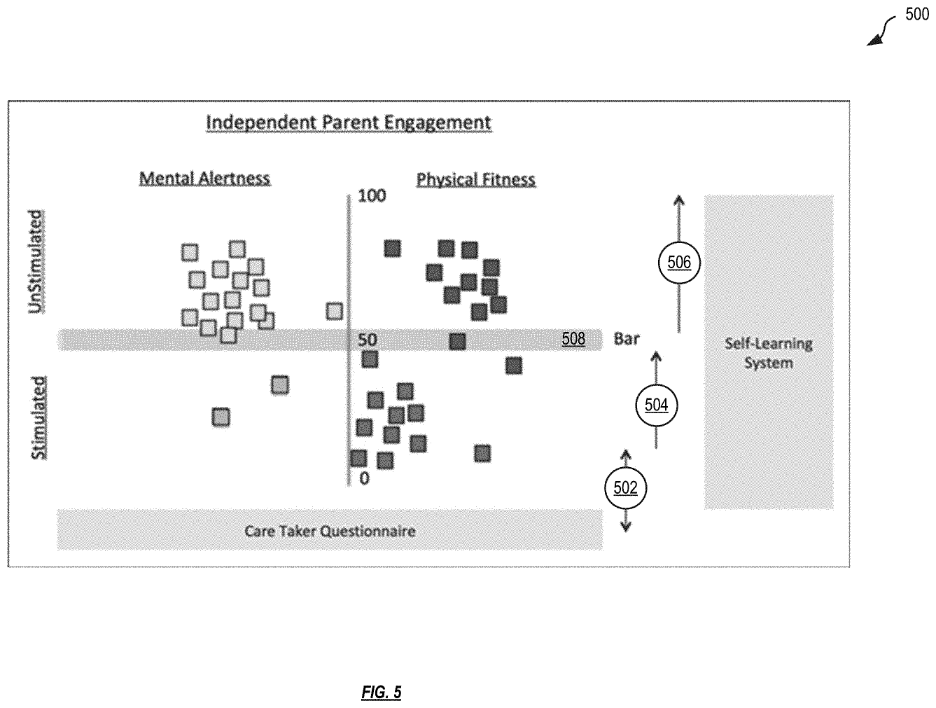

The self-learning system 456 shown in FIG. 4 evolves via implementation of a machine learning algorithm (e.g., machine learning algorithm 130, above) during the conditioning and continued-learning stages and guides the monitored individual 404 through the three monitored-individual participation phases shown as stages 502, 504, and 506, respectively in FIG. 5. FIG. 5 is a chart 500 showing monitored individual interaction with the system according to the multi-parameter response function 451, in an example. Stage 502 represents the initial configuration stage based on the questionnaire 451. Stage 504 represents the conditioning stage discussed above in which the monitored individual 404 is prompted and rewarded to utilize the wearable device 420 and/or other devices in the hardware ecosystem 458. Stage 506 represents the continued learning stage in which the self-learning system 456 continues to modify the multi-parameter response function based on the monitored individual 404's continued and changing use of the system. The goal of the self-learning system 456 is to move the monitored individual's interaction above the ‘bar’ 508 indicated in FIG. 5, which is a threshold amount of activity by the monitored individual 404 with the system, including both physical and mental fitness metrics. The bar 508 may be defined by the monitoring individual 402 during completion of the questionnaire 451.

The self-learning system 456 may further store a monitored individual behavior model 462 that utilizes additional AI interfaces 464 to be implemented by the system which will enhance the overall health and wellbeing of the monitored individual. As an example, the AI interfaces 464 may include a video interface captures a facial image once a day (or at some other interval) from the same location and position (such as in a bathroom mirror of the parent's house). These daily images over time displayed as a movie will allow a medical diagnosis Parent's wellbeing. Further, with advances in AI, and ease of third-party AI image-recognition integration, the self-learning system 456 can be notified that irregular ageing has occurred in real time.

The server 412 may collect data from other monitored individuals 404 (without sharing the data to other unauthorized monitored individuals or monitoring individuals) and continuously over time set the multi-parameter ‘bar’ 508, and also tune the monitored individual scorecard 460.

The monitored individual behavior model 462 in FIG. 4 is an example of the initial configuration settings 126 discussed above and is defined by the questionnaire 451 and modified by the actual behavior of the monitored individual 404 detected and analyzed by the system 456 during the conditioning and continued learning stages.

The hardware ecosystem shown 458 in FIG. 4 includes the wearable device 420 (which may be a GoFind, Inc. “GoFindR”) and other 3rd party devices (such as the smart speaker 122, second device 118, and additional devices 119, discussed above). The wearable device 420, which is an example of the monitor device 120, may include a hardware pendent/fob or wristband containing a cellular connection, a GPS locator, a Wi-Fi/BLE for local connectivity, accelerometer for fall and activity detection, temperature sensor, and a SOS button as well as other potential sensors as discussed above with respect to the sensor suite 206.

The hardware ecosystem 458 may provide one or more of the following features:
Geo: Locate, fencing, and vicinity
Gesture recognition: wearable: walking/shuffling/fallen/tremors
In-home 3rd party links
Home voice-based assistant
Connected home ecosystem
Environment: CO, Smoke, Temp
Health indicators: Heart rate, hydration patch, blood oxygen saturation
Computer vision and AI. Fixed-position, automated daily image capture and real-time image recognition technology may, in the future, detect accelerated ageing or early-state disease onset warnings. The same could be applied to video recognition in detecting irregularities in gestures like walking/shuffling/fallen, as well as body limb tremors in the sitting or standing still position.

During the conditioning and/or continued learning stages, the self-learning system 456 may prompt the monitored individual with a stimulate/reward action 466 in FIG. 4 allows an action 468 defined in the multi-parameter response function 452 to be presented to the monitored individual 404 by the self-learning System 256, such as, “It is time to wake up”. In certain embodiments, the questionnaire 451 may further include a voice capture section 470 in which, for each action 468 that requires a voice prompt from the system, the monitoring individual 402 reads a displayed prompt during the questionnaire, and the monitoring individual 402's voice is recorded (using the microphone of the device on which the monitoring individual 402 is completing the questionnaire) and stored in the self-learning system 456 in association with the action 468. Thus, when the stimulate/reward action 466 is prompted to the monitored individual 404, it is in the monitoring individual's 402 voice.

This prompt may be followed by a reward response 472, which is an example of the acknowledgment 308 discussed above. An example of response 472 is “GoFind, I'm up”, generated by the monitored individual 404 and provided to the wearable device 420, or to another device such as smart speaker 122. This process allows for a system output to monitored individual 404 via the smart speaker 122 or the device 118 (e.g., the stimulate prompt 466), to stimulate and/or reward the monitored individual 404. The system thus stimulates monitored individual 404 to invoke physical, mental or emotional activity interaction with the system by completing the response 472. When the monitored individual 404 responds, the monitored individual 404 is rewarded (e.g., the monitored individual scorecard 460 is updated) and thus conditions the monitored individual 404 to use the system via a lure-reward conditioning mechanism.

This stimulate/reward conditioning mechanism opens up a new user model where remote monitoring individuals 402 set monitored individual 404 activity goals with Internet-based rewards such as content streaming, and on-line purchases and home delivery. Moreover, because the prompts associated therewith may, in embodiments, be in the voice of the monitoring individual 402 (e.g., as collected as voice recording 129, discussed above), the prompts are more likely to be actioned on by the monitored individual 404.

If a non-response 474 occurs (shown in FIG. 4) from monitored individual 404 will generate an alert 454 (e.g., the above discussed alert 310 or no alert 312) and either initiate another Stimulate prompt 466, depending on the parameters in the Self-Learning System 256 as defined by the Multi-Parameter Response Function 452. The alert 454 is shown as an output 476 from the server 412, and the output 454 may consist of audio prompts via smart speaker 122, SMS text messages, push internet notifications, or an SOS alarm to authorities, the closest neighbor (e.g., the secondary individual 128 discussed above), or the monitoring individual(s) 402. No Response events 474 are logged in the self-learning system (such as in the monitored individual scorecard 460).

The system output 476 may include, but is not limited to, the following:
push to a Mobile Client (e.g., running on one or more of devices 116, 118, discussed above);
Web dashboard showing history of past events related to the parent 404,
Push Notifications
SMS/Texts
Incoming call from Parents
SoS Alerts
Ad-hoc Calls
SoS Alerts

At any time, an unstimulated action 478 may be detected using hardware ecosystem 458 and transmitted to the self-learning system 456. For example, the unstimulated action 478 may be a fall which was detected by the accelerometer in the wearable device 420. This unstimulated action 478 may cause generation of the alert 454, and/or stored in the parent scorecard 460 or used to update the multi-parameter response function 452.

If the system 456 identifies that a given event is an emergency, the output 476 may indicate such to monitoring individual 402 (or other entity as identified as the secondary individual 128 discussed above) and also uses third-party home voice-based assistant to contact the monitored individual 404 if they are in the home and within the vicinity of Speaker with microphone determined by the wearable device 420. The monitoring individual 402 may also call monitored individual 420, and vice-versa, directly hands-free, and have a phone voice conversation via a direct voice communication channel 480 established therebetween.

The phone voice conversation 480 in FIG. 4 may require that the parent 404 be home and in the vicinity of a voice-activated call device (such as the wearable device 420, a phone, or a smart speaker 122 discussed above). Table 2 below indicates potential instances of automatic initiation, by system 256, of the voice communication channel 480.

TABLE 2

| Mode of Operation | Caller | Receiver |
|---|---|---|
| System-detected | System initiates: | Caregiver(s) Group |
| SoS Alert | Push notification | together with system- |
| | SMS Text | determined Admin or |
| | In-home voice- | ‘nearest neighbor' member |
| | controlled device | of Caregiver(s) Group. |
| Parent voice- | Parent is in SoS state, | Pre-set Caregiver(s) |
| activated | and voice activates | Admin or closest member |
| SoS Alert | system if in vicinity | of the Caregiver(s) group |
| | of fixed, in-home | |
| | voice-activated call | |
| | device | |
| Ad-hoc Parent-> | Parent voice-activated | Caregiver(s) receives via: |
| Caregiver call | call to Caregiver(s) | Mobile Client; or |
| | if in vicinity of | In-home, fixed |
| | fixed, in-home | voice-activated call |
| | voice-activated call | device. |
| | device | |
| Ad-hoc Caregiver-> | Caregiver(s) call to | Parent hears/sees incoming |
| Parent call | Parent via: | call if in vicinity of fixed, |
| | Mobile Client; or | in-home voice-activated |
| | In-home, fixed | call device. |
| | voice-activated call | |
| | device. | |

Third-party Cloud Services 482 in FIG. 4 is similar to the third-party server 132 discussed above, and allows for 3rd party cloud services managed by Google, Amazon or Apple, for example, to be the hardware interface, which prompts Parent 404 in the voice of her or his Caregiver(s) 402. Third party cloud services 482 includes, but is not limited to, one or more of the following:
Hardware ecosystem, including Voice Interface/Recognition
Artificial Engines and Predictive Health Analytics data pipelines
Automated pick-up, transport, and delivery service.

The System 256 structures its internal real-time data for use with third-party AI/deep learning and computer vision machines. Advancements in AI and deep learning machines can use the daily activity/images/video data from the individual monitored individual 404 and an aggregate of all monitored individuals 404 activity with the system 256 to predict the onset of accelerated aging and/or early-stage onset of disease.

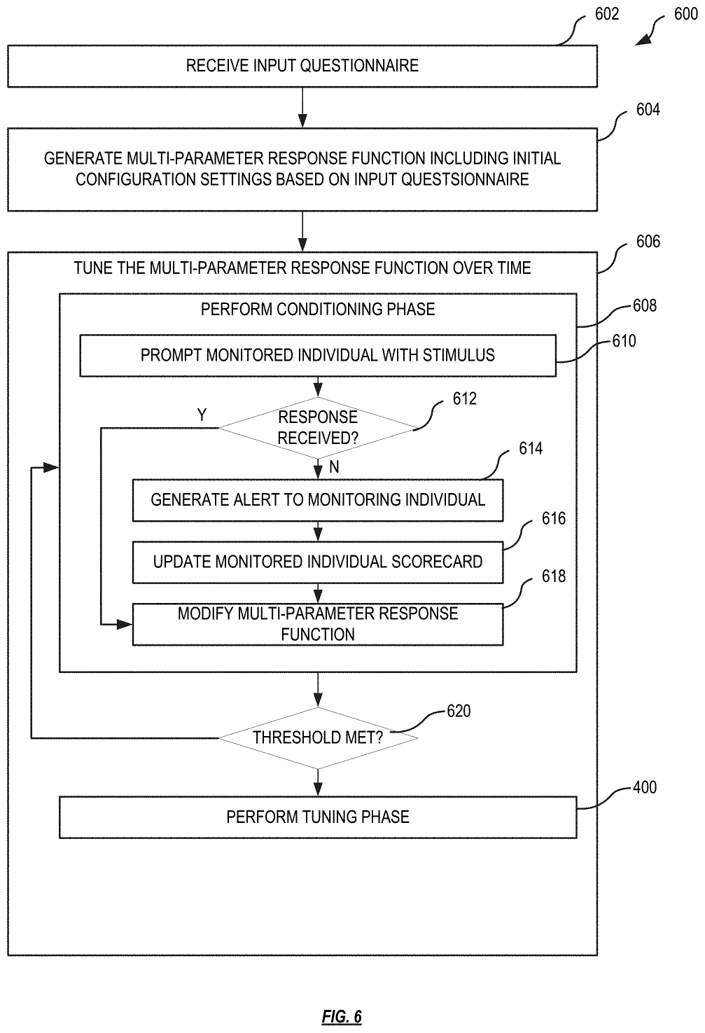

FIG. 6 depicts a method 600 for monitoring an individual at a remote location, in an embodiment. Method 600 is implemented using the system 100 of FIGS. 1-3, and the use case 400 shown in FIGS. 4-5. For example, the method 600 is implemented via execution of computer readable instructions by one or more processors of the server 112 and/or 412, discussed above.

In block 602, method 600 receives an input questionnaire. In one example of block 602, the server 112 receives the configuration questionnaire 124. In one example use case of block 602, the server 412 receives questionnaire 451.

In block 604, the method 600 generates a multi-parameter response function including initial configuration settings based on the received input questionnaire. In one example of block 604, the server 112 generates configuration settings 126 based on the questionnaire 124. In one example use case of block 604, the server 412 generates the multi-parameter response function 452 having initial configuration settings based on the received questionnaire 451. The generated configuration settings 126 and/or multi-parameter response function 451 may be stored on the serve 112, 412, and/or transmitted to the monitor device 120, second device 112, additional devices 119, smart speaker 122, and/or wearable device 420.

In block 606, the method 600 tunes the multi-parameter response function over time. In one example of block 606, the server 112 implements the machine learning algorithm 130 to tune the configuration settings 126 based on the monitored individual 104's interaction with one or more of the monitor device 120, second device 118, additional devices 119, and smart speaker 122. In one example of block 606, the server 412 tunes the multi-parameter response function 451 based on the monitored individual 404's interaction with one or more of the monitor device 420, second device 118, additional devices 119, and smart speaker 122.

Block 606 may include sub-blocks for implementing the tuning of the multi-parameter response function. In block 608, the method 600 implements a conditioning phase to condition the monitored individual to utilize components of the hardware ecosystem used to monitor the monitored individual. Block 608 may include sub-blocks 610-618. In block 610, method 600 prompts the monitored individual with a stimulus prompt. In one example of block 610, the monitored individual is prompted via one or more of the monitor device 420, second device 118, additional devices 119, and smart speaker 122 in accordance with activity 302 and associated configuration settings 126. In another example of block 610, stimulate prompt 466 is presented to the monitored individual 404. The prompt of block 610 may include voice recording 129, 470 if included in the questionnaire received at block 602.

In block 612, the method 600 determines if a response from the monitored individual is received in response to the stimulus prompt. In one example of block 612, the server 112 determines if the monitored individual 104 responds to the activity 302 according to acknowledgment 308. In one example of block 612, the monitored individual 404 generates response 472. If yes at block 612, method 600 proceeds with block 618, else method proceeds with block 614 (if included), or block 616 (if included), or block 618.

In block 614, the method 600 generates an alert to the monitoring individual. In one example of block 614, the server 112 generates the alert 310 defined in the configuration settings 126. In another example of block 614, the server 412 generates the alert 454.

In block 616, the method updates a monitored individual scorecard. In one example of block 616, the server 412 updates the monitored individual scorecard 460.

In block 618, the method 600 modifies the generated multi-parameter response function. In one example of block 618, based on the received response, or no response, the server 112 modifies one or more of activities 302, time 304, frequency 306, acknowledgment 308, alert 310, and no alert 312 based on auto-learn 314 settings. In another example of block 618, the server 412 modifies the multi-parameter response function 452 based on the received response 472 or no response 474.

In block 620, the method 600 determines if an interaction threshold is met. In one example of block 620, the server 412 determines if the monitored individual 404 is interacting with the system sufficiently above threshold 508. If yes, then method 600 proceeds with block 622, else method 600 continues the conditioning phase 608.

In block 622, method 600 continues to tune the multi-parameter response function based on continued use, or lack thereof, of the monitored individual with the hardware components of the monitoring system. In one example of block 622, the server 112 continues to modify the configuration settings 126 according to auto-learn settings 314 in response to the monitored individual 104's use of one or more of the monitor device 120, second device 118, additional devices 119, and smart speaker 122. In one example of block 622, the server 412 continues to modify the multi-parameter response function 452 according to the monitored individual 404's use of one or more of the monitor device 420, second device 118, additional devices 119, and smart speaker 122.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. A server for monitoring remotely located individuals, comprising:
a processor, and
memory storing computer readable instructions that, when executed by the processor operate to control the server to:
receive an input questionnaire completed by a monitoring individual,
generate initial configuration settings for a multi-parameter response function defining intended interaction of a monitored individual with hardware ecosystem at a location of the monitored individual, and
tune the multi-parameter response function over time in response to interaction of the monitored individual with the hardware ecosystem.

2. The server of claim 1, wherein said receive an input questionnaire includes receiving at least one voice recording in of voice the monitoring individual.

3. The server of claim 2, the multi-parameter response function including an action prompt including the voice recording.

4. The server of claim 1, wherein said tune the multi-parameter response function includes implementing a conditioning stage including:
outputting a stimulus prompt to the monitored individual, and
rewarding the monitored individual in response to receipt of a response by the individual to the stimulus prompt.

5. The server of claim 4, wherein said tune the multi-parameter response function includes implementing a self-tuning stage including:
monitoring un-prompted interactions with the hardware ecosystem by the monitored individual, and
modifying the multi-parameter response function based on the un-prompted interactions

6. The server of claim 5, the self-tuning stage occurring in response to the monitored individual interaction level passing a predetermined threshold.

7. The server of claim 6, the predetermined threshold being identified in the input questionnaire.

8. The server of claim 6, the predetermined threshold being based on data collected at the server defining interaction levels of other monitored individuals.

9. The server of claim 1, wherein said tune the multi-parameter response function includes implementing a self-tuning stage including:
monitoring un-prompted interactions with the hardware ecosystem by the monitored individual, and
modifying the multi-parameter response function based on the un-prompted interactions.

10. The server of claim 1, said instructions further controlling the server to maintain a monitored individual scorecard defining interactions and non-interactions by the monitored individual in response to prompts generated by the server.

11. The server of claim 10, the monitored individual scorecard further defining progress towards a generic and/or monitoring individual-set reward.

12. The server of claim 10, the monitored individual scorecard being accessible by the monitored individual via a web- and/or mobile-application.

13. A method for monitoring a monitored individual, comprising:
receiving an input questionnaire from a monitoring individual device;
generating configuration settings of a multi-parameter response function for a wearable device worn by the monitored individual; and,
tuning the multi-parameter response function over time in response to interaction of the monitored individual with the hardware ecosystem.

14. The method of claim 13, wherein said receiving an input questionnaire includes receiving at least one voice recording in of voice the monitoring individual.

15. The server of claim 14, wherein said tuning the multi-parameter response function over time includes prompting the monitored user with an action defined in the multi-parameter response function using the voice recording.

16. The server of claim 13, wherein said tuning the multi-parameter response function over time includes implementing a conditioning stage including:
outputting a stimulus prompt to the monitored individual, and
rewarding the monitored individual in response to receipt of a response by the individual to the stimulus prompt.

17. The server of claim 13, wherein said tuning the multi-parameter response function over time includes implementing a self-tuning stage including:
monitoring un-prompted interactions with the hardware ecosystem by the monitored individual, and
modifying the multi-parameter response function based on the un-prompted interactions

18. The server of claim 17, the self-tuning stage occurring in response to the monitored individual interaction level passing a predetermined threshold.

19. The server of claim 13, further comprising maintaining a monitored individual scorecard defining interactions and non-interactions by the monitored individual in response to prompts generated by the server.

20. The server of claim 19, the monitored individual scorecard further defining progress towards a generic and/or monitoring individual-set reward.
